(19) United States
(12) Patent Application Publication
(10) Pub. No.: US 2010/0070026 A1
(43) Pub. Date: Mar. 18, 2010

(54) STENT WITH POROUS MEMBRANE AND MANUFACTURING METHOD THEREOF

(75) Inventors: Koju ITO, Minami-ashigara-shi (JP); Kentaro SHIRATSUCHI, Minami-ashigara-shi (JP); Hidekazu YAMAZAKI, Minami-ashigara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/559,151

(22) Filed: Sep. 14, 2009

(30) Foreign Application Priority Data
Sep. 12, 2008 (JP) 2008-234917

(51) Int. Cl. A61F 2/06; B05D 3/00

(52) U.S. Cl. 623/1.39; 623/1.42; 427/2.24

(57) ABSTRACT

A dipping bath contains a polymer solution. Stent body members are dipped into the polymer solution. The polymer solution forms membrane on the surface of the stent body member. Humid atmosphere is created around the stent body members with the membrane to condense water vapor into water droplets on the surface of the membrane. After growing the water droplets to water drops, a solvent is evaporated, and the water drops penetrate into the membrane. Then, the water drops are evaporated with leaving pores in the membrane. The water drops function as templates.

### BACKGROUND OF THE INVENTION

1. Field of the Invention

The present invention relates to a stent that opens a narrowed blood vessel from inside and is implanted within the blood vessel to keep the blood vessel open, and a manufacturing method of the stent. The present invention especially relates to the stent having porous membrane on its surface and a manufacturing method thereof.

2. Description Related to the Prior Art

A medical instrument called stent is widely used for the treatment of stenosis, strictures and aneurysms in blood vessels. The stent of tubular shape is implanted within the blood vessel such as a coronary artery to keep the artery open.

For the purpose of preventing in-stent restenosis, a covered stent has been developed. The covered stent consists of a metal stent body (bare-metal stent) and polymer membrane with many pores that covers the bare-metal stent (refer to Japanese Patent Laid-Open Publication Nos. 11-299901 and 2005-152004). A drug-eluting stent has been also developed in which polymer membrane has the function of gradually eluting a biologically active agent.

According to Japanese Patent Laid-Open Publication No. 11-299901, after a bare-metal stent is covered with polymer membrane, a laser punches pores in the polymer membrane. Laser punching, however, is nonproductive, and unsuitable for forming the fine pores with a few micrometers pitch.

According to Japanese Patent Laid-Open Publication No. 2005-152004, a bare-metal stent is covered with porous membrane. This reference has the advantages of improvement in productivity and ease of forming the fine pores with a few micrometers pitch because the pores are formed in the polymer membrane in advance. However, since the porous membrane covers the peripheral surface of a stent body in a quiescent state at the time of manufacture, radial expansion of the stent body when used stretches the porous membrane. The stretch enlarges the pores and deforms the pores into an oval shape, and hence necessary porous structure disappears. Accordingly, the effect of preventing the in-stent restenosis deteriorates.

Any of the stents described above needs a process of covering the stent body with the membrane. There are stent bodies of various sizes as the variety of body parts of a lumen in which the stent is implanted. The stent bodies have a length of 2 to 3 mm and an outer diameter of 0.5 to 1 mm at the minimum, and a length of 40 mm and an outer diameter of 4 mm at the maximum. Since the largest stent body is still small in size, the covering process requires high-precision processing technique. Thus, covering the stent body with the membrane interferes with productivity improvement.

### SUMMARY OF THE INVENTION

An object of the present invention is to provide a method for efficiently manufacturing a stent with porous membrane.

Another object of the present invention is to provide the stent that can restrain deformation of pores and peeling of the porous membrane by expansion of a stent body when used.

According to the present invention, a method for manufacturing a stent with a porous membrane includes the steps of coating a surface of a stent body with a liquid that contains a polymer and a hydrophobic solvent to form a membrane having water drops, evaporating the hydrophobic solvent so that the water drops get into the membrane, and evaporating the water drops with leaving a plurality of pores in the membrane. The water drops functions as templates.

The liquid may contain a curative drug.

The coating step may include the steps of applying the liquid to the stent body to form the membrane on the surface of the stent body, condensing water vapor into water droplets on the surface of the membrane, and growing the water droplets to the water drops. An atmosphere with the water vapor has a higher dew point than a surface temperature of the membrane.

The liquid may be applied to the stent body by dipping.

Otherwise, the coating step may include the steps of putting the stent body into the liquid, forming water droplets on a surface of the liquid, raising the stent body out of the liquid having the water droplets, and growing the water droplets to the water drops. The liquid forms the membrane, and the water droplets are arranged on the membrane.

A stent with a porous membrane according to the present invention includes a stent body, and the porous membrane on a surface of the stent body. The porous membrane has a plurality of pores that are formed by water drops functioning as templates.

The porous membrane may contain a curative drug.

According to the present invention, since the membrane is directly formed on the stent body by dipping, the stent body is smoothly expanded in a radial direction. Since the porous membrane covers the entire surface of the stent body down to, for example, all stent wires, the porous membrane can smoothly follow the expansion of the stent body when used, and hence it is possible to restrict the collapse and deformation of the pores, as compared with a conventional stent. Thus, the function of preventing the in-stent restenosis does not deteriorate. After the wet membrane is formed, the porous membrane is formed by drying the membrane with the use of the water drops as the templates. Therefore, the present invention does not require a high-precision process for covering the stent body with the membrane, which was conventionally necessary, so that it is possible to efficiently manufacture the stent with the porous membrane.

### BRIEF DESCRIPTION OF THE DRAWINGS

For more complete understanding of the present invention, and the advantage thereof, reference is now made to the following descriptions taken in conjunction with the accompanying drawings, in which:

FIG. 1A is a front view of a stent in a quiescent state;

FIG. 1B is a sectional view of the stent taken along line B-B of FIG. 1A;

FIG. 10 is a front view of the stent in an expanded state;

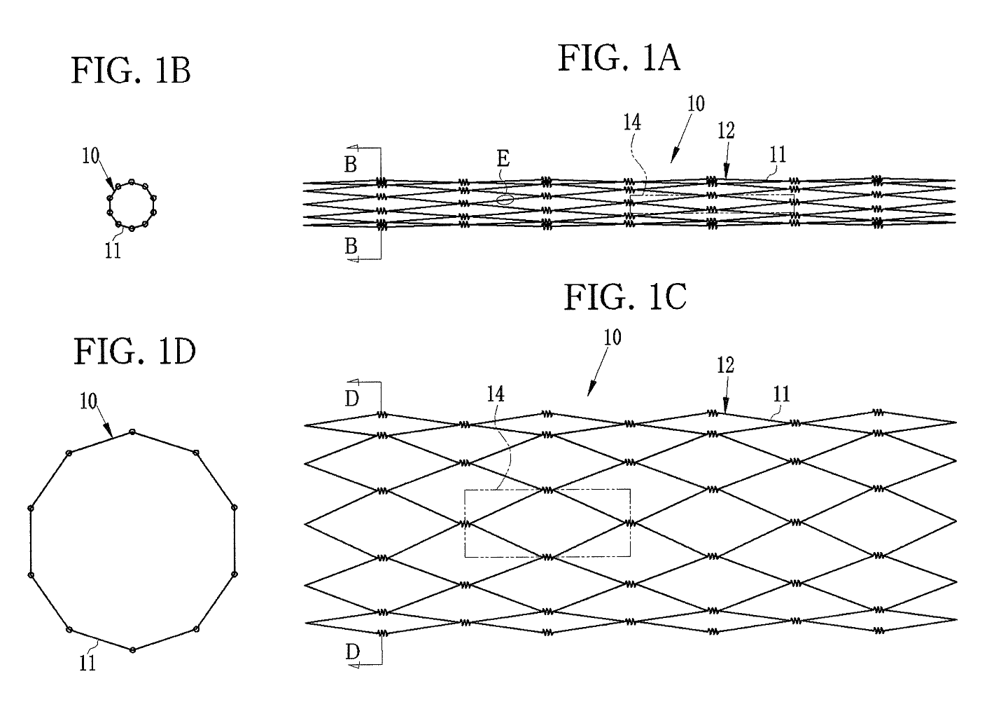

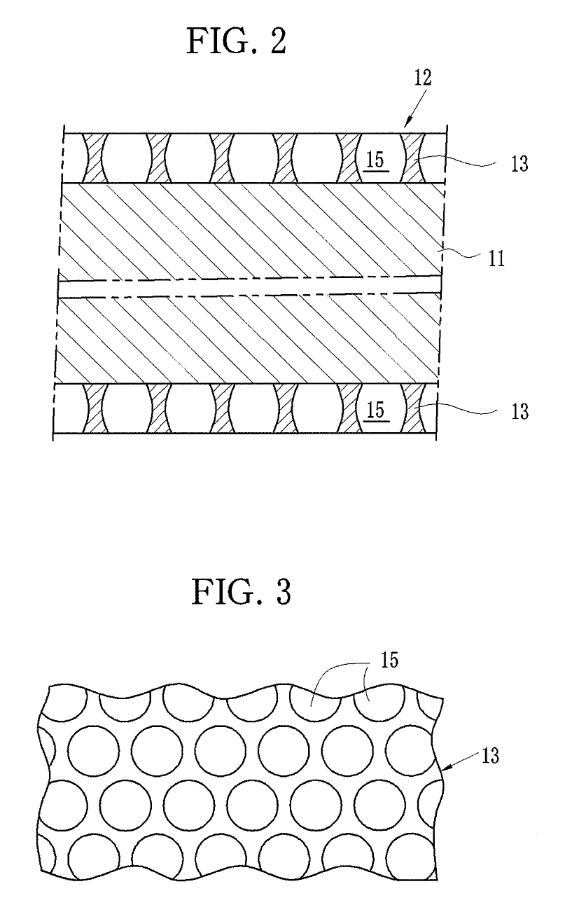

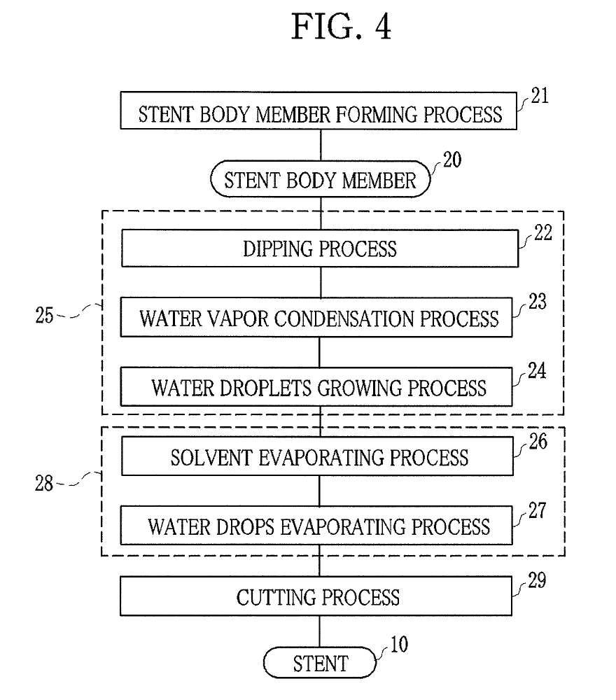

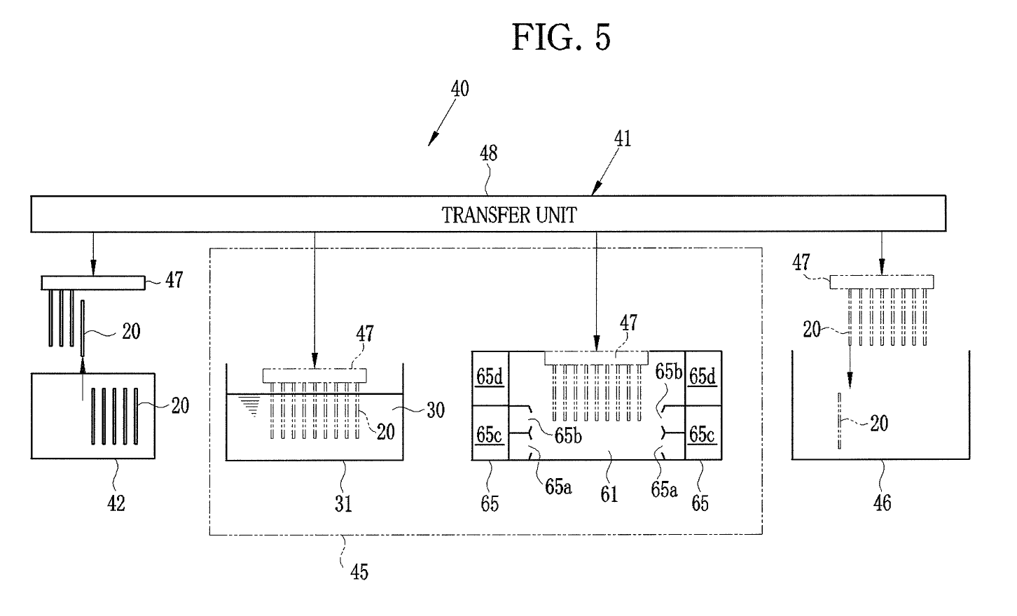

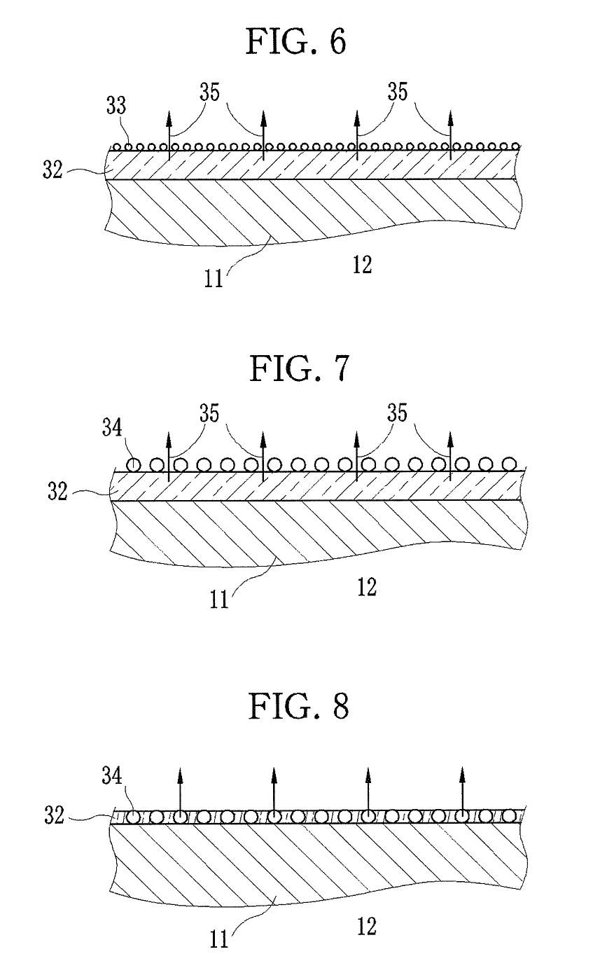

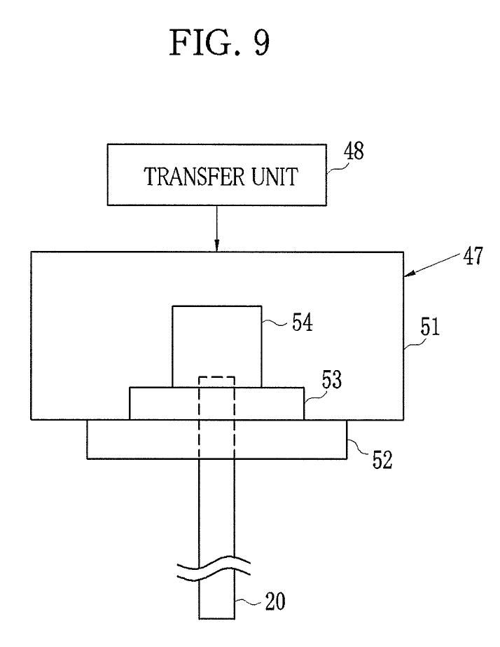

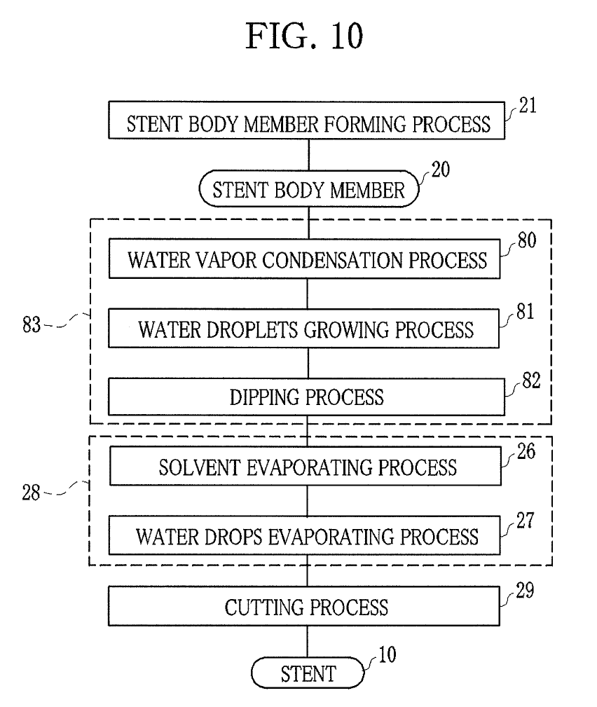

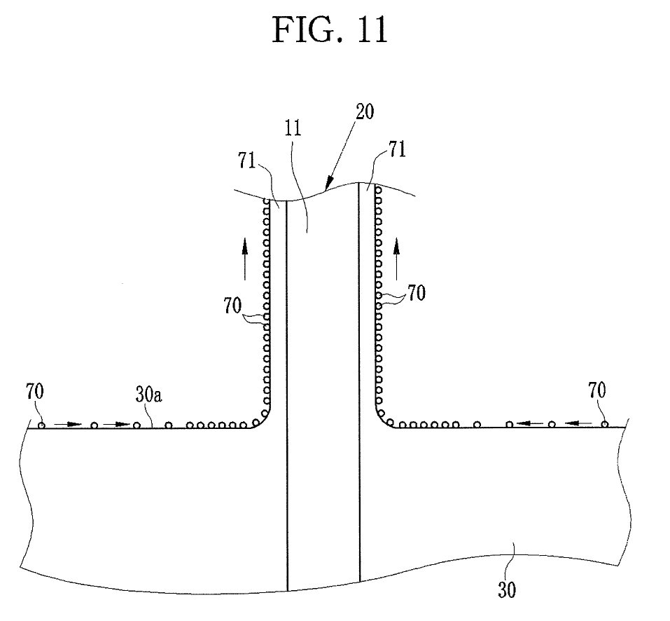

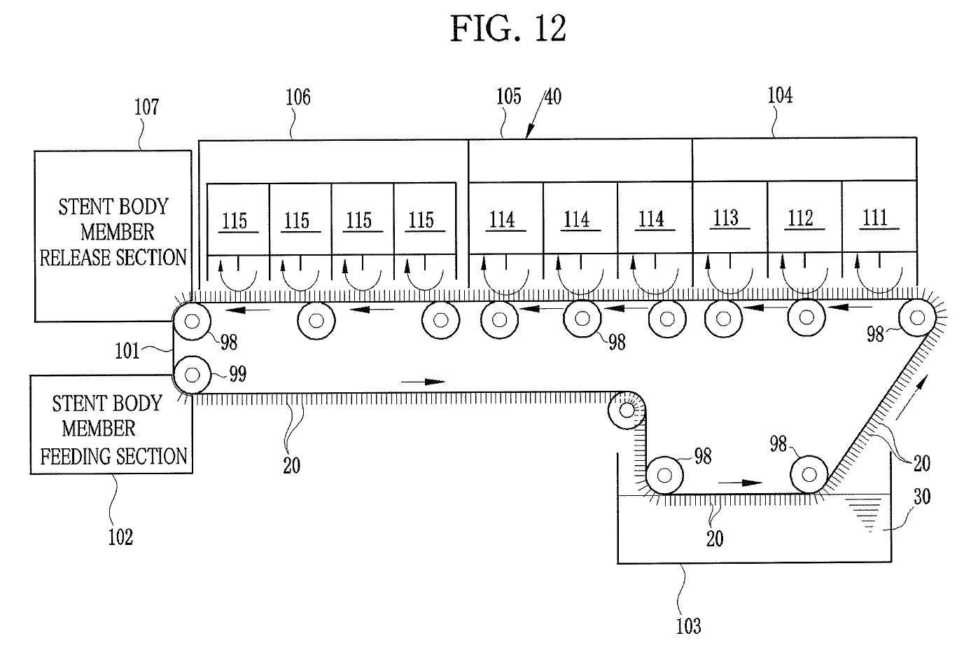

FIG. 1D is a sectional view of the stent taken along line D-D of FIG. 10;

FIG. 2 is a schematic sectional view of an area E of FIG. 1A;

FIG. 3 is an enlarged plan view of porous membrane;

FIG. 4 is a flowchart of a stent manufacturing method according to a first embodiment;

FIG. 5 is a schematic view of a stent manufacturing apparatus;

FIG. 6 is an explanatory view of a waver vapor condensation process;

FIG. 7 is an explanatory view of a waver droplets growing process;

FIG. 8 is an explanatory view of a drying process;

FIG. 9 is a schematic view of a holding plate;

FIG. 10 is a flowchart of a stent manufacturing method according to a second embodiment;

FIG. 11 is an explanatory view of a dipping process; and

FIG. 12 is a schematic view of a stent manufacturing apparatus for continuously manufacturing stents.

### DESCRIPTION OF THE PREFERRED EMBODIMENTS

As shown in FIGS. 1A to 1D, a stent 10 is constituted of a cylindrical stent body 12 that is made of stent wires 11 in an open lattice structure, and porous membrane 13 (refer to FIG. 2) for covering the surface of the stent wires 11. The stent body 12 contains a plurality of extension elements 14, which is made of the stent wire 11 shaped into a rhombus or a loop. The extension elements 14 are joined in the circumferential and longitudinal directions of the stent body 12 into a mesh, and constitute the stent body 12. The stent body 12 is expandable in a radial direction, and is deformable from a quiescent state shown in FIGS. 1A and 1B to an expanded state shown in FIGS. 1C and 1D.

The shape and material of the stent body 12 are not especially limited as long as the stent body 12 is small in diameter and is deformable between the quiescent state and the expanded state. The stent 10 in the quiescent state is easy to carry over a lumen such as a blood vessel to a lesion, and the stent 10 in the expanded state opens and supports the lumen. The stent body 12 may be constructed of plates instead of the stent wires 11, and may be made of any material such as metal and polymer. Japanese Patent Laid-Open Publication No. 2005-152004 describes the shape and material of the stent body 12 in detail.

As shown in FIGS. 2 and 3, the porous membrane 13 has planar honeycomb structure in which approximately spherical pores 15 with a roughly constant diameter are arranged in a hexagonal close-packed manner in a plane. The porous membrane 13 may instead have multi-layer honeycomb structure in which the approximately spherical pores 15 with the roughly constant diameter are arranged in a hexagonal close-packed three-dimensional manner. The pore 15 does not have to be a perfect sphere, and may be a part of a sphere as shown in FIG. 2. Part of the pores 15 may be coupled to another adjoining pore 15.

Referring to FIG. 4, a stent manufacturing method according to a first embodiment broadly includes a stent body member forming process 21, a membrane with water drops forming process 25, a drying process 28, and a cutting process 29. The membrane with water drops forming process 25 includes a dipping process 22, a water vapor condensation process (water droplets forming process) 23, and a water droplets growing process 24. The drying process 28 includes a solvent evaporating process 26 and a water drops evaporating process 27.

A stent body member 20 is manufactured in the stent body member forming process 21. The stent body member 20 has a length of plural stent bodies 12, for example, two to fifty stent bodies 12. The stent body 12 may be used instead of the stent body member 20, and the porous membrane 13 may cover the surface of the stent body 12. The stent body 12 and the stent body member 20 are manufactured by a conventional method, and the structure thereof is not especially limited.

In the dipping process 22, the stent body member 20 is dipped into a polymer solution. A dipping means is not limited as long as the minute cylindrical stent body member 20 is coated with the polymer solution. For example, as shown in FIG. 5, a dipping bath 31 retains a polymer solution 30, and the stent body members 20 are dipped into the polymer solution 30. Dip coating is preferably used in forming membrane 32 (refer to FIG. 6) on the stent body member 20. By the dip coating, the membrane 32 can be uniformly formed on the stent wires 11 complexly assembled.

In the water vapor condensation process 23, as shown in FIG. 6, water vapor condenses into water droplets 33 on the surface of the membrane 32 formed on the stent wires 11.

In the water droplets growing process 24, as shown in FIG. 7, the water droplets 33 formed on the surface of the membrane 32 grow to water drops 34. The water drops 34 are arranged in the hexagonal close-packed manner.

In the solvent evaporating process 26, as shown in FIG. 7, a solvent 35 evaporates from the membrane 32. Thus, the water drops 34 penetrate into the membrane 32 as shown in FIG. 8, while the membrane 32 is dried.

In the waver drops evaporating process 27, as shown in FIG. 8, the water drops 34 evaporate from the membrane 32 with leaving pores in the membrane 32. The water drops 34 function as templates. The membrane 32 with the pores is identical to the porous membrane 13 formed on the surface of the stent wires 11, as shown in FIGS. 2 and 3.

In the cutting process 29, the stent body member 20 is cut into a plurality of stent bodies 12. Thus, the stent 10 with the porous membrane 13 is manufactured. Instead of cutting, the stent body member 20 may be subjected to another processing such as compressing.

The porous membrane 13 is made of a thermoplastic, elastic and/or bioabsorbable polymer that is chosen in terms of stretchability for enabling ease of stretch and not interfering with expansion of the stent body 12. Examples of preferable non-biodegradable polymers include metallocene-catalyzed polyolefins, vinyl aromatic polymers such as polystyrene, vinyl aromatic copolymers typified by styrene-isobutylene copolymers, polyethylene vinyl acetate (EVA), polyvinyl chloride (PVC), fluorinated polymers, polyester, polyamide, polyether, polyurethane, polysilicone, polycarbonate, and mixtures and copolymers of any of all the above. The metallocene-catalyzed polyolefins include polyethylene, polypropylene, polybutylene, polybutadiene, polyisobutylene and copolymers thereof. The vinyl aromatic copolymers include styrene-isobutylene-styrene (preferably, TRANSLUTE (trademark) made by Boston Scientific), butadiene-styrene copolymers and other block copolymers.

Examples of preferable biodegradable polymers include polylactic acid such as poly(L-lactide) (PLLA) and poly(D,L-lactide) (PLA), polyglycolic acid [polyglycolide (PGA)], and copolymers and mixtures thereof. The preferable biodegradable polymers further include poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), poly(D,L-lactide-co-caprolactone) (PLA/PCL), poly(glycolide-co-caprolactone) (PGA/PCL), polyethylene oxide (PEO), polydioxanone (PDS), polypropylene fumarate, poly(ethylglutamate-co-glutamic acid), poly(tert-butyloxy-carbonylmethyl glutamate), polycaprolactone (PCL), polycaprolactone-co-butyl acrylate, polyhydroxybutyrate (PHBT), poly(phosphazene), poly(phosphate ester), poly(amino acid), poly(hydroxybutyrate), polydepsipeptide, maleic anhydride copolymers, polyimino carbonate, poly[(97.5% dimethyl-trimethylene carbonate)-co-(2.5% trimethylene carbonate)], cyanoacrylate, polysaccharides such as methylcellulose, ethylcellulose and acetylcellulose, and mixtures and copolymers of any of all the above. It is preferable that the weight-average molecular weight of the above polymers be 5,000 to 1,000,000, and more preferably be 10,000 to 500,000.

Any amphiphilic polymer is available as long as the polymer is nontoxic to a living body. To be more specific, the amphiphilic polymers preferably include polyethylene glycol-polypropylene glycol block copolymers; amphiphilic polymers having a main chain of acrylamide polymers, a hydrophobic side chain of a dodecyl group and a hydrophilic side chain of a lactose group or a carboxyl group; ion complexes of an anionic polymer such as heparin, dextran sulfate, or a nucleic acid including DNA and RNA and long chain alkyl ammonium salt; and amphiphilic polymers having a hydrophilic group of water-soluble protein such as gelatin, collagen and albumin. Due to the superior function of stabilizing the water drop 34 being the template, amphiphilic polymers including dodecyl acrylamide-w-carboxyhexyl acrylamide are especially preferable.

Any organic solvent is available as long as the solvent dissolves hydrophobic and macromolecular compounds. Examples of the organic solvent include aromatic hydrocarbon (e.g. benzene and toluene), hydrocarbon halide (e.g. dichloromethane, chlorobenzene, carbon tetrachloride and 1-bromopropane), cyclohexane, keton (e.g. acetone and methyl ethyl keton), ester (e.g. methyl acetate, ethyl acetate and propyl acetate) and ether (e.g. tetrahydrofuran and methyl cellosolve). Is also available a compound of a pure substance or mixture of the above components with adding a small amount of hydrophilic solvent such as alcohol or keton by approximately 20% or less. In the case of not using dichloromethane, ether with 4 to 12 carbon atoms, ketone with 3 to 12 carbon atoms, ester with 3 to 12 carbon atoms, hydrocarbon bromine such as 1-bromopropane or the like is preferably used for the purpose of minimize environmental impact. A mixture of these chemical agents may be used instead. For example, a mixed organic solvent of methyl acetate, acetone, ethanol and n-butanol is available. These ether, ketone, ester and alcohol may have cyclic structure. A compound having any two or more functional groups of ether, ketone, ester and alcohol (that is, —O—, —CO—, —COO— and —OH—) is available as the solvent. When the solvent is a compound of two or more chemical agents different from each other, appropriately varying the ratio of the agents makes it possible to control the speed of forming water drops 34, the depth of penetration of the water drops 34 into the membrane 32 and the like.

As for the membrane, it is preferable that the polymer be between or equal to 0.02 and 30 parts by weight relative to 100 parts by weight of the organic solvent. This ratio facilitates forming the good-quality porous membrane 13 with high productivity. When the polymer does not reach 0.02 parts by weight relative to 100 parts by weight of the organic solvent, the ratio of the solvent is too large in the solution. It takes long time to evaporate the solvent, so that the productivity of the porous membrane 13 becomes worse. When the polymer exceeds 30 parts by weight, on the other hand, the condensed water drops 34 cannot deform the membrane 32, so that the porous membrane 13 may be uneven.

In the case of using a mixture of a polymer and an amphiphilic compound, it is preferable that the weight of the amphiphilic compound is between or equal to 0.1% and 20% of the weight the macromolecular compound, because the formed water drops 34 tend to be uniform in size and hence the porous membrane 13 with uniform pores is obtained. When the weight of the amphiphilic compound is less than 0.1% of the weight of the macromolecular compound, the added amphiphilic compound has little effect. Thus, the formed water drops 34 may be unstable and nonuniform in size. When the weight of the amphiphilic compound having low-molecular-weight occupies more than 20% of the weight of the macromolecular compound, on the other hand, the strength of the porous membrane 13 may be reduced.

When the porous membrane according to the present invention contains a biologically active agent (curative drug), the active agent is dissolved in the polymer solution before forming the membrane 32. Thus, the formed porous membrane 13 contains the active agent. Besides dissolving the active agent in the polymer solution, the active agent may be applied to the surface of the membrane 32 after being formed.

The active agent includes at least one compound chosen among groups of an anticancer drug, an immunosuppressive drug, an antibiotic, an anti-rheumatic drug, an antithrombotic drug, an HMG-CoA reductase inhibitor, an ACE inhibitor, a calcium antagonist, an antilipidemic agent, an integrin inhibitor, an antiallergic agent, an antioxidant agent, a GPIIb/IIIa antagonist, retinoid, flavonoid, carotenoid, an anti-lipid drug, a DNA synthesis inhibitor, a tyrosine kinase inhibitor, an antiplatelet agent, an anti-inflammatory agent, a living body-derived material, interferon and a NO producing agent.

It is preferable that the diameter of the spherical pore 15 of the porous membrane 13 be 0.1 to 100 μm, more preferably 0.1 to 50 μm, and most preferably 0.1 to 25 μm. When the pore 15 takes the shape of part of a sphere, the diameter of the spherical pore 15 refers to the maximum diameter of the sphere in a direction orthogonal to thickness of the porous membrane 13. Forming the pores 15 within these confines allows sufficient exchange of substances between an inner surface and an outer surface, when the stent 10 with the porous membrane 13 in the honeycomb structure is implanted in the lesion. Accordingly, the stent 10 accelerates endothelialization of the inner wall of the blood vessel and prevents in-stent restenosis.

It is preferable that the surface of the porous membrane 13 according to the present invention accelerate the growth of endothelial cells. To be more specific, when the porous membrane contains, for example, the amphiphilic polymer, the hydrophilic group of the amphiphilic polymer tends to make a chemical bond to a blood vessel endothelial cell precursor, so that the growth of the endothelial cells is accelerated. Doping polyethyleneglycol on the surface of the porous membrane 13 is also preferable to accelerate the growth of the endothelial cells.

As shown in FIG. 5, a stent manufacturing apparatus 40 according to the present invention has a handling section 41, a feeding section 42, a membrane forming and drying section 45 and a release section 46. The handling section 41 has holding plates 47 and a transfer unit 48. The holding plate 47 holds a plurality of stent body members 20. The transfer unit 48 successively transfers the holding plates 47 from the feeding section 42 to the membrane forming and drying section 45 and the release section 46.

Referring to FIG. 9, the holding plate 47 has a plate body 51, clampers 52, thermoregulators 53 and ejectors 54. The clamper 52 catches and holds an end of the stent body member 20. The thermoregulator 53 regulates the temperature of the stent body member 20 held by the clamper 52 to keep surface temperature of the membrane 32 within a certain range, as described later on. The ejector 54 releases the catch of the clamper 52 to detach the stent body member 20 from the holding plate 47 after the porous membrane 13 is formed on the surface of the stent body member 20.

As shown in FIG. 5, a general-purpose transfer unit having a rail, a guide mechanism, a robot arm and the like is available as the transfer unit 48 as long as the unit can send the holding plates 47 to the feeding section 42, the membrane forming and drying section 45 and the release section 46.

In this embodiment, the stent body member 20 having a length of, for example, 2 to 50, preferably 5 to 20 stent bodies 12 is used for the purpose of efficiently forming the porous membrane 13. The stent body member 20 is cut into individual stent bodies 12 in the cutting process 29 (refer to FIG. 4) after the porous membrane 13 is formed.

The membrane forming and drying section 45 is provided with the dipping bath 31 and a processing chamber 61 for forming, growing and drying the water drops 34. The dipping bath 31 contains the polymer solution 30 that is kept at predetermined temperature. The transfer unit 48 pauses over the dipping bath 31, and slides down the holding plate 47. The transfer unit 48 dips the stent body members 20 into the polymer solution 30 in the dipping bath 31, and then raises the stent body members 20 up. Thus, the stent body members 20 are dip-coated with the polymer solution 30. The wet thickness of the membrane 32 in the dipping process 22 is 1 mm or less, preferably between or equal to 10 μm and 400 μm, and more preferably between or equal to 20 μm and 300 μm. The viscosity of the polymer solution 30 is between or equal to 1×10⁻⁴ Pa·s and 1×10⁻¹ Pa·s, preferably 5×10⁻⁴ Pa·s and 5×10⁻² Pa·s.

As a method to apply the polymer solution 30 to the stent body members 20, there are spraying, flow coating, brushing and the like available other than dipping. However, dipping is likely to the best way, in consideration of the open lattice structure of the stent body member 20.

After that, the transfer unit 48 transfers the holding plate 47 to the processing chamber 61. In the processing chamber 61, the water vapor condensation process (water droplets forming process) 23, the water droplets growing process 24 and the drying process 28 shown in FIG. 4 are carried out.

The processing chamber 61 is provided with an air blower 65. The air blower 65 is provided with ducts 65c, each of which has a fan 65a and an intake port 65b, and an air controller 65d. The air controller 65d sucks air around the membrane 13 through the intake ports 65b. The air controller 65d adjusts the temperature, dew point and humidity of the air, and blows the air from the discharge ports 65a into the processing chamber 61. Accordingly, atmosphere around the stent body members 20 is circulated, and the processing chamber 61 is maintained in desired states. The ducts 65c have a filter for removing dust from the air. The processing chamber 61 may have a single or plurality of air blowers 65. The structure of the air blower 65 is not limited to the above as long as the water drops 34 are uniformly formed on the stent body members 20 held by the holding plate 47.

The air blower 65 supplies the humid air to the processing chamber 61, and water vapor condenses in the surfaces of the membrane 32. First atmosphere refers to atmosphere in which the waver vapor condenses into the water droplets 33. Creating the first atmosphere in the processing chamber 61 carries out the water vapor condensation process 23 (refer to FIG. 4). When “Td1” represents a dew point in the first atmosphere and “Ts” represents the surface temperature of the membrane 32, a value “ΔT1” calculated by “Td1−Ts” is more than zero. It is preferable that the value “ΔT1” be between or equal to 0.5° C. and 30° C. Setting the value “ΔT1” within such a range can form a lot of water droplets 33 on the surface of the membrane 32 by condensation. It is preferable to have an air velocity of between or equal to 0.05 m/s and 10 m/s.

After that, the air blower 65 changes the dew point and the like of the humid air, and creates second atmosphere in the processing chamber 61 to grow the water droplets 33 (water droplets growing process 24). When “Td2” represents a dew point in the second atmosphere and “Ts” represents the surface temperature of the membrane 32, a value “ΔT2” calculated by “Td2−Ts” is more than zero. It is preferable that the value “ΔT2” be between or equal to 0.5° C. and 20° C. Setting the value “ΔT2” within such a range can grow the water droplets 33 on the surface of the membrane 32. ΔT1>ΔT2 is generally preferable in terms of uniformity. It is preferable to have an air velocity of between or equal to 0.05 m/s and 10 m/s. The thermoregulator 53 provided in the holding plate 47 regulates the temperature of the stent body member 20 and hence the surface temperature “Ts” of the membrane 32. The thermoregulator 53 may be provided in the clamper 52 or the plate body 51. The dew point “Td” is controlled by changing the conditions of the humid air blown from the air blower 65.

When the water droplets 33 become water drops 34 of a desired size, as shown in FIG. 7, the air blower 65 changes the temperature, humidity and dew point of blowing air, and creates third atmosphere around the membrane 32 of the stent body members 20 to carry out the solvent evaporating process 26 (refer to FIG. 4). In the third atmosphere, the solvent in the membrane 32 evaporates, and the water drops 34 penetrate into the membrane 32. The water drop 34 in the membrane 32 functions as a template. The third atmosphere has such temperature that the solvent in the membrane 32 evaporates and the water drops 34 form the templates. In the third atmosphere, the temperature of the blowing air is between or equal to 5° C. and 50° C., and an air velocity is between or equal to 0.05 m/s and 10 m/s, for example.

Next, by changing the dew point and the like of the humid air, fourth atmosphere is created to carry out the water drops evaporating process 27. The water drops 34 evaporate and leave minute pores in the membrane 32. Thus, the porous membrane 13 is formed. In the fourth atmosphere, the temperature of the blowing air is between or equal to 5° C. and 100° C., and an air velocity is between or equal to 0.01 m/s and 20 m/s, for example.

The transfer unit 48 transfers the dried stent body members 20 to the release section 46. The stent body members 20 are ejected from the holding plate 47 in the release section 46. The stent body members 20 are sent to the cutting process 29, and a not-illustrated cutter cuts the stent body members 20 into predetermined lengths to form the stents 10. Thus, the stents 10 with the porous membrane 13 are manufactured without a coating process and the like.

As described above, a successive shift of atmosphere inside the processing chamber 61 from the first atmosphere to the fourth atmosphere carries out the membrane with water drops forming process 25 including the water vapor condensation process 23 and the water droplets growing process 24 and the drying process 28 including the solvent evaporating process 26 and the water drops evaporating process 27. The water droplets 33, the water drops 34 and the membrane 32 are magnified in FIGS. 6 to 8 for the purpose of clarification.

A dipping chamber having the dipping bath 31 and the processing chamber 61 are provided with a not-illustrated solvent recovery system to recover the solvent. A not-illustrated recycle system recycles the recovered solvent.

Although manufacturing efficiency degrades, the holding plate 47 may hold the stent bodies 12 and carry out dipping. In this case, a grip is provided at a tip of the stent body 12 for ease of handling, and a clamp, hook or the like holds the grip. After the porous membrane 13 is formed, the grip is cut off from the stent body 12. Thus, the porous membrane 13 covers the entire surface of the stent body 12. In a like manner, the stent body member 20 may be provided with a grip, and the holding plate 47 may detachably hold the grip.

The thickness (wet thickness) of the membrane 32 before drying is between or equal to 0.01 mm and 1 mm. Even if the thickness of the membrane 32 is within this range, there may be cases where the water drops 34 cannot be uniformly formed due to unevenness of the membrane 32. When the wet thickness is less than 0.01 mm, the membrane 32 cannot be formed uniformly. There are cases where a part of the stent wire 11 sheds the polymer solution 30, and the membrane 32 does not cover the entire surface of the stent wire 11. When the wet thickness exceeds 1 mm, on the other hand, drying the membrane 32 requires long time and reduces manufacturing efficiency. Also, the too thick membrane 32 is not suitable for the stent 10.

In the water vapor condensation process 23, the air blower 65 blows humid air on the membrane 32 through the fans 65a, and sacks air around the membrane 32 from the intake ports 65b so as to create the first atmosphere in the processing chamber 61. By controlling at least one of the surface temperature “Ts” and the dew point “Td1”, the value “ΔT1” calculated by “Td1−Ts” is between or equal to 0.5° C. and 30° C., wherein “Td1” represents the dew point of the humid air from the fans 65a, and the “Ts” represents the surface temperature of the membrane 32.

In the second atmosphere in the water droplets growing process 24, humid air is blown in a like manner. The value “ΔT2” calculated by “Td2−Ts” is between or equal to, for example, 0.5° C. and 20° C., wherein “Td2” represents the dew point of the humid air from the fans 65a in the second atmosphere. By setting the value “ΔT2” within such a range, the water droplets 33 grow on the surface of the membrane 32. In terms of uniformity, “ΔT2” is set smaller than “ΔT1”. To measure the surface temperature “Ts” of the membrane 32, for example, a commercial noncontact thermometer such as an infrared thermometer is provided in the plate body 51.

In the solvent evaporating process 26, the third atmosphere is created in the processing chamber 61 to evaporate the solvent. The grown water drops 34 penetrate into the membrane 32, and function as the templates for forming the pores 15. After that, the fourth atmosphere is created in the processing chamber 61 to evaporate the water drops 34. Accordingly, it is possible to obtain the porous membrane 13 with the pores 15 that have a diameter of between or equal to 0.1 μm and 50 μm and are arranged in the honeycomb structure.

In the second atmosphere, the thermoregulator 53 can control the surface temperature “Ts” of the membrane 32. Instead of or in addition to the thermoregulator 53, a temperature control plate (not illustrated) may be disposed on the plate body 51 adjacently to the clamper 52 to control the surface temperature “Ts” of the membrane 32. The dew point “Td” is controlled by changing the conditions of the humid air from the air blower 65.

In the third atmosphere, the air blower 65 blows air of, for example, an air temperature of 30° C. and an air velocity of 0.3 m/s. Leaving the water drops 34, only the solvent in the membrane 32 is evaporated.

In the fourth atmosphere, one of the surface temperature “Ts” and the dew point “Td” is controlled so as to make the surface temperature “Ts” higher than the dew point “Td”. The thermoregulator 53 controls the surface temperature “Ts”. The air blower 65 controls the dew point “Td” of air from the fans 65a. The thermometer measures the surface temperature “Ts”. Setting the surface temperature “Ts” higher than the dew point “Td” makes it possible to stop growing the water drops 34 and evaporate the water drops 34 to form the porous membrane 13. In the case of Ts≦Td1, water vapor may further condense on the membrane 32 with the water drops 34 and destroy formed porous structure. The fourth atmosphere is intended for evaporating the water drops 34, but the remaining solvent is also evaporated. In the fourth atmosphere, the air blower 65 blows air of, for example, an air temperature of 30° C. and an air velocity of 2 m/s.

In the water drops evaporating process 27 in the fourth atmosphere, a vacuum dryer or the so-called 2D nozzle may be used instead of the air blower 65. The vacuum dryer allows easy regulation of the evaporation rate of the solvent and the water drops 34 individually. Thus, the organic solvent evaporates more preferably, and the water drops 34 preferably penetrate into the membrane 32. The vacuum dryer assists to form the pores in regulated positions, size and shape. In the 2D nozzle, a plurality of fan nozzle sections for blowing air and a plurality of intake ports for sacking air around the membrane 32 are alternately disposed in an arrangement direction of the stent body members 20. Otherwise, a drying chamber may be provided separately from the processing chamber 61 to carry out the water drops evaporating process 27 therein.

Referring to FIG. 10, in a membrane with water drops forming process 83 according to a second embodiment, water droplets are formed on a surface 30a of the polymer solution 30 (a water vapor condensation process 80 of FIG. 10). After that, the water droplets are grown to water drops 70, and the water drops 70 are arranged in a hexagonal close-packed manner on the solution surface 30a (a water droplets growing process 81). Then, the stent body members 20 sank in the polymer solution 30 are raised out of the polymer solution 30 that remains in a state of closely arranging the water drops 70 on the solution surface 30a (a dipping process 82), so that the membrane 71 having the water drops 70 on its surface is formed. The same reference numbers as the first embodiment refer to the same, identical or similar processes and components and description thereof is omitted.

In FIG. 10, the dipping process 82 consists of a stent body member putting process for putting the stent body members 20 into the polymer solution 30, and a stent body member raising process for raising the stent body members 20 out of the polymer solution 30. The stent body member putting process of the dipping process 82 may be carried out before the water vapor condensation process 80.

FIG. 11 shows the membrane with water drops forming process 83 according to the second embodiment. The water drops 70 are formed on the surface 30a of the polymer solution 30 in the water vapor condensation process 80 and the water droplets growing process 81. Then, the stent body members 20 are raised out of the polymer solution 30 having the water drops 70, so that the membrane 71 with the water drops 70 is formed on the surface of the stent body member 20.

The water drops 70 are illustrated largely in FIG. 11. The water drops 70 in the hexagonal close-packed manner are formed in a small area of the solution surface 30a in FIG. 11, but are actually formed in a larger area. On the solution surface 30a, there are a lot of water drops 70 that are enough for being supplied to the surfaces of the stent wires 11. The raise of the stent body members 20 and evaporation of the polymer solution 30 cause a solution flow in the surface 30a of the polymer solution 30. The solution flow makes the water drops 70 collect in a raise position by advection.

In the water vapor condensation process 80, a first humid air supplier supplies first humid air to the surface 30a of the polymer solution 30 to form the water droplets on the surface 30a by condensation. A method of forming the water droplets is the same as that of the first embodiment. In the water droplets growing process 81, a second humid air supplier supplies second humid air to the surface 30a of the polymer solution 30 to grow the water droplets by condensation. A method of growing the water droplets is the same as that of the first embodiment.

In the second embodiment, as in the case of the first embodiment, after the membrane with the water drops forming process 83, the solvent is evaporated with leaving the water drops 70 (solvent evaporating process 26), and then the water drops 70 are evaporated (water drops evaporating process 27). By carrying out the drying process 28 like this, a lot of pores are formed in the membrane 32 with the use of the water drops 70 functioning as templates. After the stent body members 20 are raised from the polymer solution 30, the stent body members 20 may be subjected to the water droplets growing process 24, as with the first embodiment, to grow the water drops 70 larger. In this case, a porous membrane with pores of a larger diameter can be formed.

In the first and second embodiments, the water droplets are formed on the polymer solution 30 by condensation, but the water droplets may be ejected on the membrane 32 or the surface 30a of the polymer solution 30 by an inkjet method.

In the first and second embodiments, all of the water vapor condensation process 23, the water droplets growing process 24, the solvent evaporating process 26 and the water drops evaporating process 27 are carried out in the single processing chamber 61 by changing conditions such as humidity and temperature. However, separate processing and drying chambers may be provided for individual processes instead.

The foregoing embodiments adopt a batch method, and the stent body members 20 are subjected to each process on a holding plate 47 basis. A continuous production method in which holding plates 47, as shown in FIG. 9, are attached to an endless transfer system such as a belt conveyer and a chain conveyer may be adopted instead.

FIG. 12 shows a stent manufacturing apparatus 100 according to the continuous production method. The stent manufacturing apparatus 100 is provided with an endless belt 101 as a transfer unit. To the endless belt 101, holding plates 47 are attached at regular intervals. The holding plate 47 is identical to that shown in FIG. 9, and has a clamper 52, an ejector 54, a thermoregulator 53 and the like. In the holding plate 47 shown in FIG. 9, the clamper 52 holds the stent body member 20 one by one. A plurality of stent body members 20 may be attached to a holding block in advance, and the clamper 52 may hold the holding block instead.

Guide rollers 98 and a drive roller 99 decide a transfer route of the endless belt 101. Along the transfer route, a stent body member feeding section 102, a polymer solution dipping bath 103, first to third processing chambers 104 to 106 and a stent body member release section 107 are arranged in this order in a belt movement direction. Except for continuously moving the stent body members 20, stent manufacturing procedure is the same as that of the first embodiment.

The stent body member feeding section 102 feeds the stent body members 20 to the holding plates 47 (refer to FIG. 9), and clamps a plurality of stent body members 20 to the holding plates 47. The clamped stent body members 20 are successively sent to the polymer solution dipping bath 103, the first to third processing chamber 104 to 106 and the stent body member release section 107 in this order with movement of the endless belt 101.

In the dipping process, the stent body member 20 proceeds in the polymer solution 30 contained in the polymer solution dipping bath 103, and is then raised. The stent body member 20 is coated with the polymer solution 30. The stent body member 20 coated with the polymer solution 30 is sent to the first processing chamber 104. In the first processing chamber 104, the water vapor condensation process and the water droplets growing process are carried out. The first processing chamber 104 is provided with first to third air blowers 111 to 113. Each of the air blowers 111 to 113 basically has the same structure as the air blower 65 of the first embodiment.

The first air blower 111 blows air to create the first atmosphere around the stent body members 20. Thus, water droplets 33 are formed on the surface of the membrane 32 of the stent body members 20 by condensation. The second and third air blowers 112 and 113 blow air to create the second atmosphere around the stent body members 20. Thus, the water droplets 33 grow to the water drops 34 on the surface of the membrane 32 of the stent body members 20.

The second processing chamber 105 is provided with fourth air blowers 114. The fourth air blower 114 also has the same structure as the air blower 65 of the first embodiment. The fourth air blowers 114 create the third atmosphere around the stent body members 20. Thus, the solvent evaporates from the membrane 32 of the stent body members 20.

The third processing chamber 106 is provided with four fifth air blowers 115. The fifth air blower 115 also has the same structure as the air blower 65 of the first embodiment. The fifth air blowers 115 create the fourth atmosphere around the stent body members 20. Thus, water drops 34 evaporate from the membrane 32 of the stent body members 20.

In the stent body member release section 107, the ejector 54 ejects the stent body member 20 with the porous membrane 13 from the holding plate 47. The ejected stent body member 20 is cut into predetermined lengths by a not-illustrated cutter to obtain the stents 10 with the porous membrane 13.

### Practical Example 1

Next, the practical example 1 of the present invention will be described. In the stent manufacturing apparatus 40 shown in FIG. 5, the membrane with water drops forming process 25 was carried out. After that, the solvent and the water drops 34 were evaporated by the drying process 28 to form the porous membrane 13. The stent body member 20 had an outer diameter of 2 mm and a length of 30 mm. As the dipped polymer, a dichloromethane solution 2.5 mg/ml of a polystyrene-polyisoprene-polystyrene block copolymer is used.

According to this example, it was possible to almost uniformly manufacture porous membrane 13 in which the diameter of pores was 3.0 μm, the pitch of adjoining pores 15 was 4.0 and the thickness was 1.5 μm. It was verified that the manufacturing method according to the present invention could reduce peeling of the porous membrane 13 and deformation of the pores 15 in expanding the stent body 12, in contrast to a conventional stent in which porous membrane covered the periphery of the stent body after pores were formed in the membrane.

The present invention does not require a porous membrane forming process for forming porous membrane and a covering process for covering the stent body with the porous membrane, which were necessary in a conventional manufacturing method. Conventionally, covering a small cylindrical stent, which had an outer diameter of 0.5 to 4 mm and a length of approximately 40 mm, with porous thin membrane of a thickness of 1 to 50 μm required a high-precision covering process. In the present invention, however, since the porous membrane 13 is formed directly on the stent body 12, it is possible to manufacture the stent 10 with ease.

Appropriately regulating conditions of forming and growing the water droplets 33 makes it possible to easily change the diameter and the pitch of the pores 15 of the porous membrane 13. In the conventional method, the preferable thickness of the porous membrane was 1 to 10 μm due to the limit of adhesion of the porous membrane. The present invention, however, offers superior adhesion between the surface of the stent body 12 and the porous membrane 13, so that thickness limitation is relieved. Namely, the superior adhesion brings about reduction in the thickness of the membrane 13. Therefore, it is possible to provide the stent 10 in which the porous membrane 13 is hard to peel off the stent body 12 and that is easily transferred to the lesion.

Although the present invention has been fully described by the way of the preferred embodiment thereof with reference to the accompanying drawings, various changes and modifications will be apparent to those having skill in this field. Therefore, unless otherwise these changes and modifications depart from the scope of the present invention, they should be construed as included therein.

What is claimed is:

1. A method for manufacturing a stent having a porous membrane comprising the steps of:
coating a surface of a stent body with a liquid containing a polymer and a hydrophobic solvent, said liquid forming a membrane, a surface of said membrane having water drops;
evaporating said hydrophobic solvent from said membrane and said water drops getting into said membrane; and
evaporating said water drops with leaving a plurality of pores in said membrane, said water drops functioning as templates.

2. The method according to claim 1, wherein said liquid contains a curative drug.

3. The method according to claim 1, wherein said coating step comprising the steps of:
applying said liquid to said stent body, said liquid forming said membrane on said surface of said stent body;
condensing water vapor into water droplets on said surface of said membrane, an atmosphere with said water vapor having a higher dew point than a surface temperature of said membrane; and
growing said water droplets to said water drops.

4. The method according to claim 3, wherein said liquid is applied to said stent body by dipping.

5. The method according to claim 1, wherein said coating step comprising the steps of:
putting said stent body into said liquid;
forming water droplets on a surface of said liquid;
raising said stent body out of said liquid having said water droplets, said liquid forming said membrane, said water droplets being arranged on said membrane; and
growing said water droplets to said water drops.

6. A stent with a porous membrane comprising:
a stent body; and
said porous membrane on a surface of said stent body, said porous membrane having a plurality of pores that are formed by water drops functioning as templates.

7. The stent according to claim 6, wherein said porous membrane contains a curative drug.
